(19) United States
(12) Patent Application Publication
(10) Pub. No.: US 2009/0043344 A1
(43) Pub. Date: Feb. 12, 2009

(54) METHODS FOR REPAIRING DEFECTS IN BONE

(75) Inventor: Ryan D. Schlotterback, Ft. Wayne, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER, INC., Warsaw, IN (US)

(21) Appl. No.: 11/834,211

(22) Filed: Aug. 6, 2007

(51) Int. Cl. A61F 5/00; A61B 17/58; A61M 31/00; A61F 2/28; A61B 19/00

(52) U.S. Cl. 606/86 R; 604/509; 128/898; 623/23.48

(57) ABSTRACT

A method for repairing defects in bones that may be used to remove a surface defect from the articulating surface of a bone. In one embodiment, a passage is formed in the bone and extending to an articulating surface of the bone, resulting in the removal of bone stock from the bone. By aligning the passage to intersect with the defect in the bone, the creation of the passage itself results in the removal of the defect from the articulating surface of the bone. A biocompatible material may then be inserted through the passage to replace the removed bone stock and may be formed to substantially replicate the shape of the articulating surface of the bone.

### BACKGROUND

1. Field of the Invention

The present invention relates to methods for repairing defects in bones.

2. Description of the Related Art

Articular joints, such as the hip and knee joints, are comprised of two, opposing bones that articulate relative to one another. If one of the bones of an articulating joint becomes damaged, a person may experience pain during joint articulation. For example, a surface defect, such as a focal defect, may occur in the articulating surface of one of the bones forming the joint. The surface defect may be severe enough that the resulting pain requires the person to undergo a total joint arthoplasty.

As an alternative to performing a total joint arthoplasty, the exterior of the damaged bone may be resurfaced. In order to resurface a bone forming an articulating joint, the joint is exposed and the bones forming the joint are separated. For example, to repair a defect on the surface of a head of a femur, the hip joint is exposed and the head of the femur removed from the joint capsule. The defective portion of the femur may then be removed and a cap, such as a metallic cover, secured to the femur. The femur is then returned to the joint capsule and repositioned adjacent to the acetabulum.

### SUMMARY

The present invention relates to methods for repairing defects in bones. In one exemplary embodiment, the present invention may be used to remove a surface defect from an articulating surface of a bone. In this embodiment, a passage is formed in the bone and extending to an articulating surface of the bone, resulting in the removal of bone stock from the bone. By aligning the passage to intersect with the defect in the bone, the creation of the passage itself results in the removal of the defect from the articulating surface of the bone. A biocompatible material may then be inserted through the passage to replace the removed bone stock and may be formed to substantially replicate the shape of the articulating surface of the bone. In one exemplary embodiment, the bone is positioned directly adjacent to the opposing bone of the joint prior to insertion of the biocompatible material. This allows for the opposing bone to act as a form, which shapes the biocompatible material to match the articulating surface of the opposing bone. In this manner, the defective portion of the bone is removed and an articulating surface substantially replicating the natural anatomical surface of the bone is created.

Advantageously, forming the passage through the bone, the need to remove the bone from the joint capsule is eliminated. As a result, the surrounding muscle or other ligamentous structures do not have to be resected to repair the defect. Further, by utilizing a passage formed within the bone itself, the need to expose the joint is eliminated. As a result, the procedure may be performed in a minimally invasive manner, allowing arthroscopes and other minimally invasive instruments to be utilized. This may reduce the recovery time of the patient and allow the surgeon to more easily and efficiently performance the underlying procedure. Furthermore, by utilizing the procedures of the present invention, a defect formed on the articulating surface of a bone of an articulating joint may be readily repaired without the need to undergo total joint arthoplasty.

In one form thereof, the present invention provides a method for resurfacing a defect in a bone, including the steps of: forming a passage in the bone extending from a non-articular surface of the bone through the bone to an articular surface of the bone, the passage providing access into a joint space between the articular surface of the bone and an opposing bone; and inserting a biocompatible material through the passage from the non-articular surface of the bone to the articular surface of the bone, the biocompatible material substantially replicating a portion of the articular surface of the bone.

In another form thereof, the present invention provides a method for resurfacing a defect in a bone, including the steps of forming a passage extending from a non-articular surface of the bone through the bone to an articular surface of the bone; positioning the articular surface of the bone in contact with an opposing bone; inserting a biocompatible material into the passage; and forming the biocompatible material against the opposing bone to shape the biocompatible material, wherein the shape of the biocompatible material substantially replicates the anatomical shape of a portion of the articular surface of the head of the bone.

In yet another form thereof, the present invention provides a method for resurfacing a defect in a bone, including the steps of: forming a passage extending from a lateral aspect of the bone to an articular surface of the bone, the passage providing access to a joint space between the articular surface of the bone and an opposing bone; and inserting a biocompatible material through the passage from the lateral aspect of the bone to the articular surface of the bone, the biocompatible material substantially replicating at least a portion of the articular surface of the bone.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a fragmentary, perspective view of a femur including a focal defect and a cross-section of an acetabulum cooperating with the femur to form a hip joint;

FIG. 2 is a fragmentary cross-section of the hip joint of FIG. 1 depicting a passage formed in the femur;

FIG. 3 is a fragmentary cross-section of the hip joint of FIG. 1 depicting a balloon and cannula positioned within the passage of the femur of FIG. 2;

FIG. 4 is a fragmentary cross-section of the hip joint of FIG. 1 depicting the balloon of FIG. 3 in an expanded position and a rod positioned adjacent thereto within the passage of the femur of FIG. 2;

FIG. 5 is a fragmentary cross-section of the hip joint of FIG. 1 depicting a dehydrated hydrogel and a rod positioned within the passage of the femur of FIG. 2;

FIG. 6 is a fragmentary cross-section of the hip joint of FIG. 1 depicting the hydrogel and rod of FIG. 5 with the hydrogel in a rehydrated state;

FIG. 7 is a fragmentary cross-section of the hip joint of FIG. 1 depicting a passage according to another exemplary embodiment formed within the femur of FIG. 1;

FIG. 8 is a fragmentary cross-section of the hip joint of FIG. 1 depicting articular cartilage and a rod positioned within the passage of the femur of FIG. 7;

FIG. 9 is a fragmentary, cross-sectional view of a hip joint depicting the passage of FIG. 7 formed in the femur and a void formed in the acetabulum; and

FIG. 10 is a fragmentary cross-sectional view of the hip joint according to FIG. 9, depicting biocompatible material positioned within the void in the acetabulum and within the passage in the femur.

Corresponding reference characters indicate corresponding parts throughout the several views. The exemplifications set out herein illustrate preferred embodiments of the invention and such exemplifications are not to be construed as limiting the scope of the invention in any manner.

### DETAILED DESCRIPTION

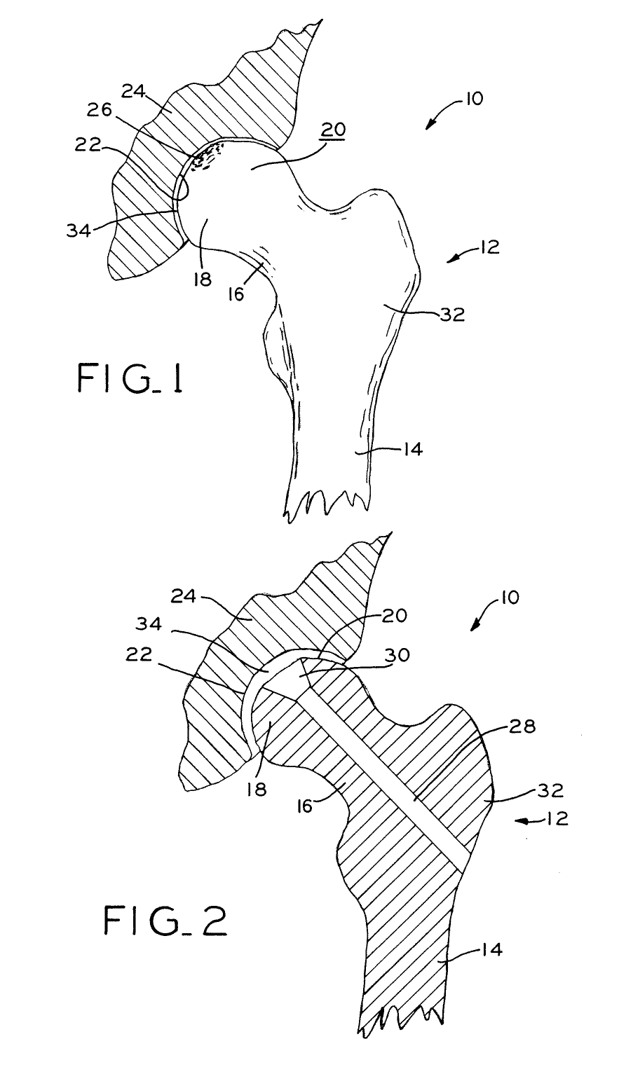

Referring to FIGS. 1-10, an articulating joint is depicted in the form of hip joint 10. While described and depicted herein with specific reference to a hip joint, the present invention may be utilized in conjunction with any articulating joint, such as a shoulder joint formed by a humerus and scapula where the head of the humerus articulates against the glenoid of the scapula, for example. Referring to FIG. 1, hip joint 10 includes femur 12 having shaft 14, neck 16, and head 18. Head 18 includes articulating surface 20 configured for articulation with corresponding articulating surface 22 of acetabulum 24. In a healthy hip joint, head 18 of femur 12 rotates within acetabulum 24 allowing for articulating surfaces 20, 22 to slide past one another. However, articulating surfaces 20, 22 may become damaged, causing a person to experience pain within hip joint 10. For example, as shown in FIG. 1, defect 26, such as a focal defect, may be formed in articulating surface 20 of head 18 of femur 12.

Referring to FIG. 2, defect 26 may be removed by forming passage 28 which is aligned to intersect with defect 26 (FIG. 1). By aligning passage 28 to intersect with defect 26, defect 26 is substantially removed during the formation of passage 28. In one exemplary embodiment, a computer assisted surgery (CAS) system, for example, a robotic surgical system or haptic device, such as described in U.S. patent application Ser. No. 11/610,728, entitled AN IMAGELESS ROBOTIZED DEVICE AND METHOD FOR SURGICAL TOOL GUIDANCE, filed Dec. 14, 2006, the disclosure of which is hereby expressly incorporated herein by reference, is utilized to facilitate the alignment of passage 28 with defect 26.

In one exemplary embodiment, shown in FIG. 2, passage 28 includes expanded portion 30 formed within head 18 of femur 12. The formation of expanded portion 30 allows for substantially all of defect 26 to be removed during the formation of passage 28 by enlarging the size of only a small portion of passage 28, i.e., the portion of passage 28 near articulating surface 20. As a result, more of the bone stock of femur 12 may be preserved. Passage 28 may be formed using a reamer, such as the reamers disclosed in U.S. patent application Ser. No. 10/721,808, entitled EXPANDABLE REAMER, filed Nov. 25, 2003 and U.S. patent application Ser. No. 11/243,7898, entitled EXPANDABLE FIXATION DEVICES FOR MINIMALLY INVASIVE SURGERY, filed Oct. 5, 2005, the entire contents of which are expressly incorporated by reference herein. In one exemplary embodiment, passage 28 is also configured to extend from a lateral aspect of femur 12, such as greater trochanter 32, to articulating surface 20 of head 18 of femur 12. By forming passage 28 extending from a lateral aspect of femur 12 through articulating surface 20 of head 18, access to joint space 34 between articulating surfaces 20, 22 is provided through passage 28.

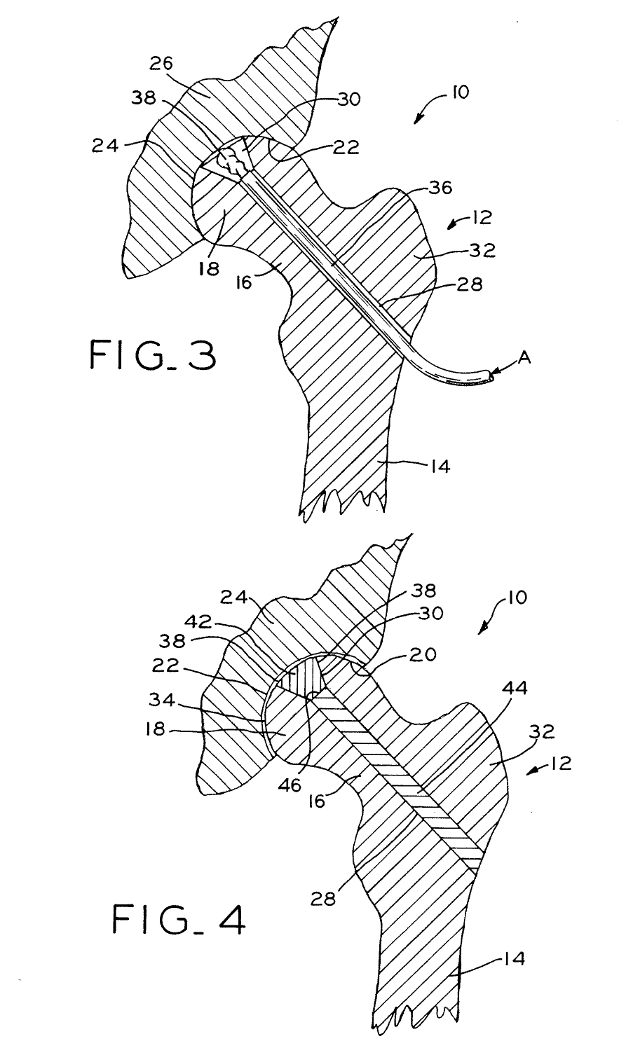

Referring to FIG. 3, cannula 36 having balloon 38 extending therefrom may be inserted within passage 28. Specifically, cannula 36 may be advanced within passage 28 to position balloon 38 within expanded portion 30 of passage 28. With cannula 36 positioned as shown in FIG. 3, femur 12 is positioned with articulating surface 20 of head 18 in direct contact with articulating surface 22 of acetabulum 24. In one exemplary embodiment, direct contact between articulating surfaces 20, 22 is achieved by a surgeon pressing head 18 of femur 12 into acetabulum 24 through manipulation of femur 12.

Biocompatible material 42 (FIG. 4), such as bone cement or an articular material, may then be injected in the direction of arrow A of FIG. 3 through cannula 36 and into balloon 38. In other exemplary embodiments, biocompatible material 42 may include or be formed of a hydrogel, saline, autograft bone, allograft bone, and/or a polymer. Additionally, biocompatible material 42 may be injected in the fluid state or be combined with a fluid prior to injection. By injecting biocompatible material 42 as a fluid, biocompatible material 42 easily passes through cannula 36 and into balloon 38. As balloon 38 is expanded by the increasing pressure of biocompatible material 42 being injected into balloon 38, articulating surface 22 acts as a form to shape balloon 38. As a result, a portion of the exterior surface of balloon 38 is shaped to substantially replicate the natural anatomical dimensions of articulating surface 20, as shown in FIG. 4. Referring to FIG. 4, after a sufficient amount of biocompatible material 42 is injected into balloon 38 to sufficiently expand balloon 38 to fill expanded portion 30, biocompatible material 42 is allowed to cure and solidify. The passage of time, exposure to ultraviolet light, or other means may be utilized to cure biocompatible material 42, rigidly securing biocompatible material 42 within expanded portion 30 of passage 28.

Further, as shown in FIG. 4, cannula 36 may be removed from balloon 38 prior to or after the curing of biocompatible material 42. To further fill passage 28 and add additional strength to femur 12, additional biocompatible material 42 may be inserted within passage 28 until passage 28 is substantially entirely filled with biocompatible material 42. In one exemplary embodiment, rod 44 (FIG. 4) may be inserted within passage 28. In one exemplary embodiment, rod 44 is sized to extend from a lateral aspect, such as greater trochanter 32, of femur 12 to end 46 of balloon 38. Rod 44 may be made at least in part of, and may be made entirely of, a highly porous biomaterial useful as a bone substitute and/or cell and tissue receptive material. A highly porous biomaterial may have a porosity as low as 55, 65, or 75 percent and as high as 80, 85, or 90 percent. An example of such a material is produced using Trabecular Metal™ technology generally available from Zimmer, Inc., of Warsaw, Ind. Trabecular Metal™ is a trademark of Zimmer Technology, Inc. Such a material may be formed from a reticulated vitreous carbon foam substrate which is infiltrated and coated with a biocompatible metal, such as tantalum, etc., by a chemical vapor deposition (“CVD”) process in the manner disclosed in detail in U.S. Pat. No. 5,282,861, entitled OPEN CELL TANTALUM STRUCTURES FOR CANCELLOUS BONE IMPLANTS AND CELL AND TISSUE RECEPTORS, the entire disclosure of which is expressly incorporated by reference herein. In addition to tantalum, other metals such as niobium, or alloys of tantalum and niobium with one another or with other metals may also be used.

Generally, the porous tantalum structure includes a large plurality of ligaments defining open spaces therebetween, with each ligament generally including a carbon core covered by a thin film of metal such as tantalum, for example. The open spaces between the ligaments form a matrix of continuous channels having no dead ends, such that growth of cancellous bone through the porous tantalum structure is uninhibited. The porous tantalum may include up to 75%-85% or more void space therein. Thus, porous tantalum is a lightweight, strong porous structure which is substantially uniform and consistent in composition, and closely resembles the structure of natural cancellous bone, thereby providing a matrix into which cancellous bone may grow to provide fixation of rod 44 in the surrounding bone of femur 12.

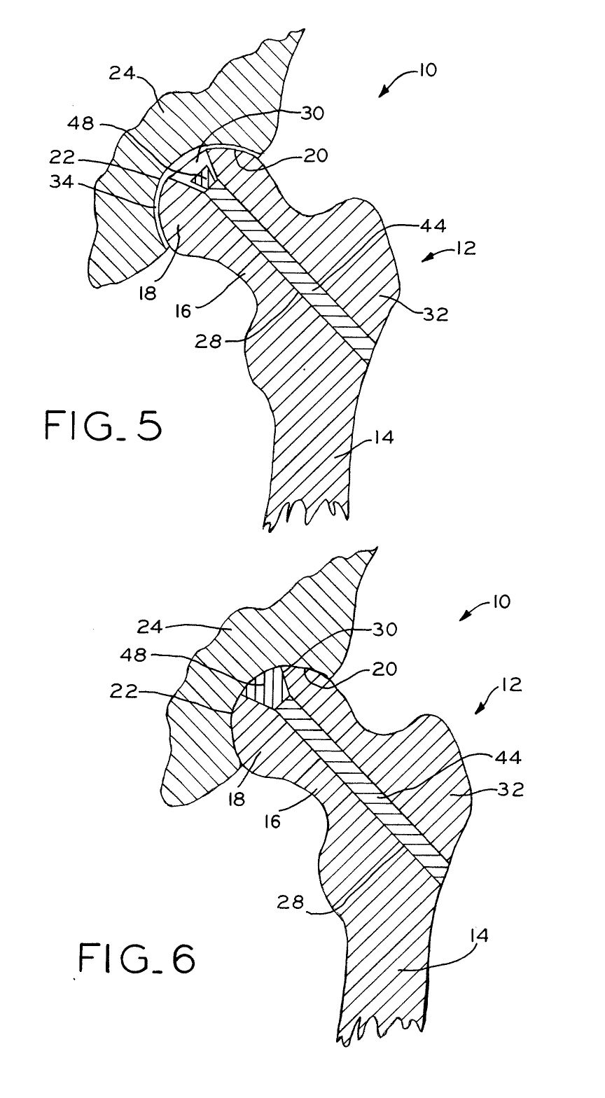

Referring to FIG. 5, another exemplary embodiment is depicted having another biocompatible material positioned within expanded portion 30. As shown, hydrogel 48 is attached to an end of rod 44 and inserted within passage 28 to position hydrogel 48 within expanded portion 30. In one exemplary embodiment, hydrogel 48 is produced using polymer material such as polyacrylates (e.g. polymethacrylate, polyhydroxyethylmethacrylate (polyHEMA), and polyhydroxypropylmethacrylate), polyvinylpyrollidone (PVP), polyvinyl alcohol (PVA), polyacrylamides, polyacrylonitriles, polysaccharides (e.g. carrageenans and hyaluronic acid), polyalginates, polyethylene oxides (e.g. polyethylene glycol (PEG) and polyoxyethylene), polyamines (e.g. chitosan), polyurethanes (e.g. diethylene glycol and polyoxyalkylene diols), and polymers of ring-opened cyclic esters. As shown in FIG. 5, hydrogel 48 has been dehydrated and, as a result, the volume of hydrogel 48 is substantially decreased. Referring to FIG. 6, hydrogel 48 is shown after rehydration within the body of a patient. Rehydration of hydrogel 48 may be facilitated by irrigating the joint or through natural absorption of fluid from the human body. Once rehydrated, hydrogel 48 expands, increasing its volume to substantially entirely fill expanded portion 30 of passage 28. Additionally, by inserting hydrogel 48 in its dehydrated form, hydrogel 48 is able to pass through the smaller portion of passage 28 and into expanded portion 30.

In one exemplary embodiment, shown in FIG. 6, articulating surfaces 20, 22 of femur 12 and acetabulum 24, respectively, are placed in contact during the rehydration of hydrogel 48. Alternatively, in another exemplary embodiment, joint space 34 is allowed to remain, creating a space between articulating surfaces 20, 22. In this embodiment, hydrogel 48 may expand beyond the natural anatomical shape of articulating surface 20 of femur 12. However, the compression of the portion of hydrogel 48 extending beyond the natural anatomical shape of articulating surface 20 of femur 12 may cause hydrogel 48 to wear down until hydrogel 48 has a shape substantially similar to the natural anatomical shape of articulating surface 20. Advantageously, the compression of hydrogel 48 may result in the release of lubricating liquid into the joint space to facilitate the articulation of femur 12 and acetabulum 24 along articulating surfaces 20, 22, respectively.

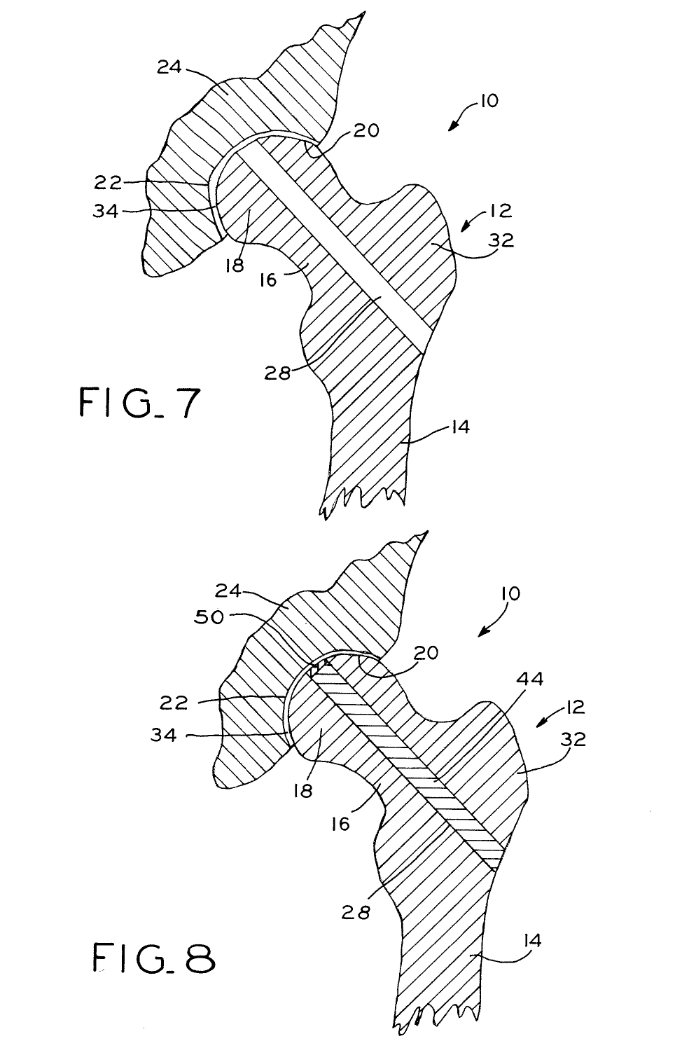

Referring to FIGS. 7-10, passage 28 is formed without expanded portion 30. In such embodiments, expanded portion 30 may be unnecessary due to a smaller size of defect 26. Alternatively, the size of passage 28 may be increased to accommodate the entirety of an enlarged defect 26 without the need for expanded portion 30. Referring to FIG. 8, rod 44 is depicted including another biocompatible material in the form of articular cartilage 50 secured thereto. Articular cartilage 50 may be a synthetic, biologics component engineered to substantially replicate the material properties of articular cartilage, for example. Alternatively, articular cartilage 50 may be articular cartilage removed from another portion of the patient's body, i.e., autograph, or may be articular cartilage removed from the body of another, i.e., allograft. Irrespective of the nature of articular cartilage 50, articular cartilage 50 may be shaped to substantially replicate the natural anatomical structure of articulating surface 20. Thus, by inserting articular cartilage 50 and, correspondingly, rod 44 into passage 28 and extending the same from the lateral aspect, for example, such as greater trochanter 32, of femur 12 to femoral head 18, articular cartilage 50 may be positioned to align with articulating surface 20 of femoral head 18 and to substantially replicate the natural anatomical shape of articulating surface 20.

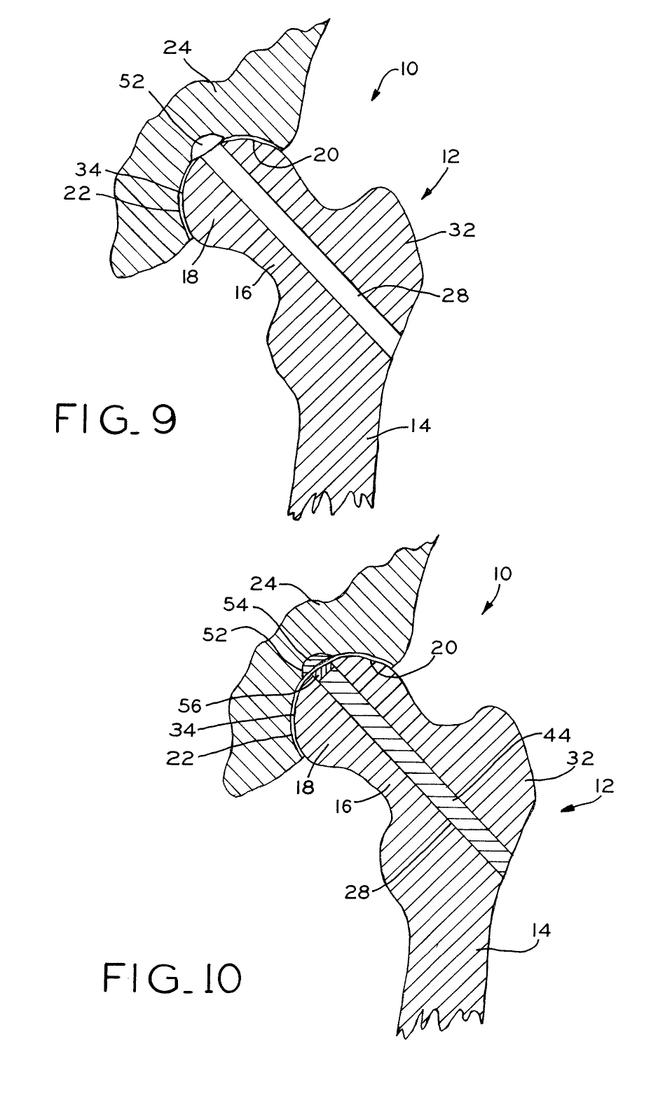

Referring to FIGS. 9 and 10, a passage may formed in one bone of a pair of articulating bones, such as passage 28 formed within femur 12, as described in detail above. Utilizing this passage, a void may be formed in the opposing bone of the pair of articulating bone. For example, referring to passage 28 formed within femur 12, void 52 may be created within acetabulum 24 to treat an acetabular defect. To form void 52 within acetabulum 24, a reamer or other bone shaping instrument, such as those described above with specific reference to passage 28, may be inserted through passage 28 to contact acetabulum 24 and form void 52. Once void 52 is formed within acetabulum 24, biocompatible material 54 may be inserted through passage 28, joint space 34, and into void 52. Biocompatible material 54 may be any of the biocompatible materials described above with specific reference to passage 28 and femur 12. For example, biocompatible material 54 may be an injectable fluid that cures to form a solid that is retained within void 52 of acetabulum 24. In one exemplary embodiment, after filling void 52, the surgeon may then rotate femur 12 to move passage 28 away from void 52 and press a portion of articulating surface 20 of femur 12 against biocompatible material 54. By pressing articulating surface 20 against biocompatible material 54, articulating surface 20 acts to shape biocompatible material 54 to substantially replicate the shape of articulating surface 22 of acetabulum 24.

Once void 52 has been filled with biocompatible material 54, passage 28 may be filled using any of the methods described herein. For example, in one exemplary embodiment, shown in FIG. 10, another biocompatible material in the form of metallic cap 56 is connected to one end of rod 44. In this embodiment, rod 44 is inserted into passage 28 and metallic cap 56 aligned with articulating surface 20. Metallic cap 56 may be configured to substantially replicate the natural anatomical shape of articulating surface 20 of femur 12. In one exemplary embodiment, metallic cap 56 is a highly polished metal, such as cobalt chrome or a titanium alloy.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method for resurfacing a defect in a bone, comprising the steps of:
forming a passage in the bone extending from a non-articular surface of the bone through the bone to an articular surface of the bone, the passage providing access into a joint space between the articular surface of the bone and an opposing bone; and
inserting a biocompatible material through the passage from the non-articular surface of the bone to the articular surface of the bone, the biocompatible material substantially replicating a portion of the articular surface of the bone.

2. The method of claim 1, wherein the biocompatible material is a curable fluid.

3. The method of claim 2, further comprising the step of inserting a balloon into the passage, wherein the step of inserting a biocompatible material into the passage further includes injecting the biocompatible material into the balloon, whereby the balloon retains the biocompatible material and expands to substantially replicate a portion of the articular surface of the bone.

4. The method of claim 1, wherein the biocompatible material comprises a rod formed from a highly porous biomaterial.

5. The method of claim 4, wherein the biocompatible material further comprises a dehyrated hydrogel attached to the rod, wherein the hydrogel is dimensioned to substantially replicate a portion of the articular surface of the bone when the hydrogel is rehydrated.

6. The method of claim 4, wherein the biocompatible material further comprises an articular material attached to the rod, the articular material selected from the group consisting of a polyethylene, a highly polished metal, and articular cartilage, wherein the articular material substantially replicates a portion of the articular surface of the bone.

7. The method of claim 1, further comprising, after the forming step, the steps of:
removing a damaged portion of the opposing bone through the passage to form a void in an articular surface of the opposing bone; and
inserting a biocompatible material through the passage and into the void to substantially replicate a portion of the articular surface of the opposing bone.

8. The method of claim 1, further comprising the step of positioning the bone directly adjacent to the opposing bone, wherein the opposing bone acts as a form to facilitate the shaping of the biocompatible material to substantially replicate a portion of the articular surface of the bone.

9. A method for resurfacing a defect in a bone, comprising the steps of:
forming a passage extending from a non-articular surface of the bone through the bone to an articular surface of the bone;
positioning the articular surface of the bone in contact with an opposing bone;
inserting a biocompatible material into the passage; and
forming the biocompatible material against the opposing bone to shape the biocompatible material, wherein the shape of the biocompatible material substantially replicates the anatomical shape of a portion of the articular surface of the bone.

10. The method of claim 9, wherein the bone comprises a femur and the opposing bone comprises an acetabulum.

11. The method of claim 10, further comprising, after the forming step, the steps of:
removing a damaged portion of the acetabulum through the passage to form a void; and
inserting a biocompatible material through the passage and into the void to substantially replicate the anatomical surface of the acetabulum.

12. The method of claim 9, wherein the bone comprises a humerus and the opposing bone comprises a glenoid.

13. The method of claim 12, further comprising, after the forming step, the steps of:
removing a damaged portion of the glenoid through the passage to form a void; and
inserting a biocompatible material through the passage and into the void to substantially replicate the anatomical surface of the glenoid.

14. The method of claim 8, wherein the biocompatible material is a curable fluid.

15. The method of claim 9, further comprising the step of inserting a balloon into the passage, wherein the step of inserting a biocompatible material into the passage further includes injecting the biocompatible material into the balloon, whereby the balloon retains the biocompatible material and expands to substantially replicate the anatomical shape of a portion of the articular surface of the bone.

16. A method for resurfacing a defect in a bone, comprising the steps of:
forming a passage extending from a lateral aspect of the bone to an articular surface of the bone, the passage providing access to a joint space between the articular surface of the bone and an opposing bone; and
inserting a biocompatible material through the passage from the lateral aspect of the bone to the articular surface of the bone, the biocompatible material substantially replicating at least a portion of the articular surface of the bone.

17. The method of claim 16, wherein the bone comprises a femur and the lateral aspect of the bone comprises a greater trochanter of the femur.

18. The method of claim 16, further comprising the step of inserting a balloon into the passage, wherein the step of inserting a biocompatible material into the passage further includes injecting the biocompatible material into the balloon, whereby the balloon retains the biocompatible material and expands to substantially replicate a portion of the articular surface of the bone.

19. The method of claim 16, wherein the biocompatible material comprises a rod formed from a highly porous biomaterial.

20. The method of claim 19, wherein the biocompatible material further comprises a dehydrated hydrogel attached to the rod, wherein the hydrogel is dimensioned to substantially replicate a portion of the articular surface of the bone when the hydrogel is rehydrated.
